(19) United States
(12) Patent Application Publication
(10) Pub. No.: US 2004/0137624 A1
(43) Pub. Date: Jul. 15, 2004

(54) Methods of site-directed transformation

(76) Inventor: Brenda A. Lowe, Mystic, CT (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/739,769

(22) Filed: Dec. 18, 2003

Related U.S. Application Data
(60) Provisional application No. 60/436,913, filed on Dec. 27, 2002.

(51) Int. Cl. C12N 15/85; C12N 15/74

(52) U.S. Cl. 435/455; 435/471

(57) ABSTRACT

Methods of integrating exogenous DNA into the genome of a eucaryotic organism comprising at least one recombination site are effected by contacting the genome with a DNA molecule comprising selected DNA and at least one site-specific recombination site which is compatible with a site-specific recombination site in the genome. DNA insertion is catalyzed by the presence of a recombinase effective for the compatible recombination sites. Selected DNA can be integrated into a genome without removal of DNA from said organism. The methods are useful for site-specific insertion of selected DNA into plants to provide fertile transgenic plants and progeny seed.

### CROSS REFERENCE TO RELATED APPLICATIONS

This application claims priority to U.S. provisional patent application serial No. 60/436,913 filed Dec. 27, 2002, which is incorporated herein by reference in its entirety.

### INCORPORATION OF SEQUENCE LISTING

The sequence listing is contained in the file named “SEQ IDs for 52823B.ST25.txt” which is 1.33 Kb (measured in MS-Windows) and was created on Nov. 13, 2003 and is located on a diskette filed herewith and incorporated herein by reference.

### BACKGROUND OF THE INVENTION

Disclosed herein are methods for site-directed transformation of eucaryotic organisms using DNA molecules with site-specific recombination sites. Also disclosed are methods of making and using such DNA molecules for site-directed transformation and recombination.

The introduction of DNA sequences into a genome has been reported for a wide variety of organisms including bacteria, viruses, yeast, plants, insects and mammals. DNA for transformation can be double-stranded DNA or single-stranded DNA, and can be circular or linear in form. Although circular double-stranded DNA is most commonly used for transformation, linear double-stranded DNA can be used as well, e.g. in microprojectile bombardment of plant cells. Linear molecules allow for transformation without undesired “ancillary” DNA which is typically contained within circular DNA molecules maintained in host cells, e.g. origins of replication, marker genes, plasmid DNA and the like.

Transformation with linear DNA molecules typically results in random integration of the DNA into the host genome and may be complex in nature, resulting in multiple copies of the inserted DNA or rearrangement of the introduced DNA. Gene expression may also vary from insertion site to insertion site, depending upon a number of factors including but not limited to, complexity or copy number of the insert, gene silencing or co-suppression, the location in the genome, the nearness of host genome regulatory sequences and the general accessibility of the genomic region to the transcription components.

Wallace et al. (Nuc. Acids Res., 28(6):1455-1464, 2000; incorporated herein by reference) and Day et al. (Genes and Dev., 14:2869-2880, 2000; incorporated herein by reference) disclose the use of site-directed integration as a method to pre-select sites in the genome for repeatable expression of transgenes in embryonic stem cells or tobacco, respectively. It is desirable in transformation technology to be able to have preselected target sites in a genome that are characterized for gene expression. Additional benefit is realized when the integrated DNA contains only the desired sequences and is targeted to a region of a genome that is known to support expression of foreign genes.

For recombinase-mediated gene replacement or gene excision, the sequence to be replaced or excised is flanked by two recombination sites. See, for example, Odell et al. (U.S. Pat. No. 5,658,772, incorporated herein by reference) which discloses the use of two loxP sites and CRE recombinase to generate specific gene replacements in tobacco. Moller et al. (WIPO Publication WO 01/40492, incorporated herein by reference) disclose the use of the CRE/lox system in an inducible manner to activate and to remove transgenes in plants. Baszczynski et al. (U.S. Pat. No. 6,187,994, incorporated herein by reference) disclose the use of multiple, non-identical frt sites and FLP-recombinase to generate a variety of gene alterations in maize. Baszczynski et al. (U.S. Pat. No. 6,262,341, incorporated herein by reference) also disclose the use of a chimeric CRE/FLP recombinase with dual target site specificity to effect recombination of DNA sequences flanked by a lox sequence on one side and a frt sequence on another side. Ow (WIPO Publication WO 02/08409, incorporated herein by reference) discloses the use of the Streptomyces phage φC31 recombination system, utilizing attB and attP sites, in gene replacement strategies in a number of organisms, including plants. The replacement or excision recombination can generate extraneous DNA fragments.

Unlike recombinase-mediated gene replacement or excision, which uses two recombination sites, site-directed integration can use a single target site in the recipient genome. Targeted, site-directed integration, where the entire exogenous DNA molecule is inserted into the target lox site is a different type of recombination event than a targeted, gene replacement integration, in which there is replacement of sequence in the host genome concurrent with an excision of sequence from the host genome. Site-specific insertion of DNA from a circular DNA template with a first single lox recombination site into a host genome with a second single lox recombination site has been reported by Albert et al. (Plant Journal, 7(4):649-659, 1995; incorporated herein by reference). In addition, Vergunst et al. (Plant Mol. Biol., 38:393-406, 1998; incorporated herein by reference) report that a pre-requisite for precise insertional recombination is a circular double-stranded DNA molecule with a single lox site.

Recently, Ow (WO02/08409; incorporated herein by reference) discloses the use of linear DNA molecules as the substrate for gene replacement using the Streptomyces phage φC31 recombination system. Ow also discloses the use of the φC31 system in conjunction with the Cre/lox system to coordinate a number of DNA manipulations including gene replacement and gene excision. In all cases, Ow used a pair of recombination sites, e.g. att sites or lox sites, for each DNA recombination event.

### SUMMARY OF THE INVENTION

This invention provides methods of adding selected exogenous DNA into the genome of a eucaryotic organism comprising at least one recombination site in its genome and where the selected exogenous DNA comprises at least one site-specific recombination site which is compatible with the recombination site in the genome of the organism. In the presence of a recombinase effective for the recombination sites, the selected exogenous DNA can be integrated into the genome at the recombination site without removal of DNA from the genome. The genome can comprise a plurality of distinct single recombination sites for multiple DNA insertions.

In one aspect, the invention provides linear DNA molecules comprising one, two, three or more site-specific recombination sites for site-directed integration into a genome without the excision of genomic DNA. In another aspect, the invention provides a circular DNA molecule comprising three or more site-specific recombination sites for site-directed integration into a genome without the excision of genomic DNA.

It is envisioned that the exogenous DNA molecules comprising one, two, three or more site-specific recombination sites may be contacted with the host cell by any method of plant transformation known to those of skill in the art, such as microparticle bombardment, Agrobacterium-mediated transformation, PEG, electroporation and the like. In a preferred embodiment, exogenous DNA is contacted to the host cell by microparticle bombardment or Agrobacterium.

Another aspect of the invention provides methods comprising identifying a transgenic recipient cell, i.e. having the selected exogenous DNA integrated into its genome. A further aspect of the invention provides methods of regenerating a transgenic organism, preferably a fertile transgenic organism capable of reproducing transgenic progeny, e.g. regenerating a fertile transgenic organism from an identified transgenic recipient cell.

The methods of the invention can employ any of a variety of recombinase/recombination site systems, e.g. the CRE recombinase/lox recombination site system, a Gin recombinase/gix recombination site system, an R recombinase/RS recombination site system, or a FLP recombinase/frt recombination site system. A preferred recombinase/recombination site system is that of the CRE recombinase/lox recombination site system.

The linear or circular DNA molecules of this invention having selected exogenous DNA and at least one recombination site can be double-stranded DNA or single-stranded DNA.

The recombinase can be provided to the linear or circular exogenous DNA and genome in a variety of ways, e.g. as a DNA molecule, as an RNA molecule or as a protein molecule. For instance, the recombinase can be provided by crossing the transgenic organism with added exogenous DNA with a second organism comprising a recombinase.

In preferred aspects of the invention, the method is used to provide a transgenic plant. Preferred aspects of the invention provide transgenic seed of fertile transgenic plants having selected exogenous DNA added into the genome without excision of native DNA.

### BRIEF DESCRIPTION OF THE DRAWINGS

FIG. 1A schematically illustrates plasmid pMON55215.

FIG. 1B schematically illustrates a derivative linear DNA molecule of pMON55215.

FIG. 1C schematically illustrates the insertion of 1mMON55215 into a host target site.

FIG. 2A schematically illustrates plasmid pMON55227.

FIG. 2B schematically illustrates a derivative linear DNA molecule of pMON55227.

FIG. 2C schematically illustrates the insertion of 1mMON55227 into a host target site.

FIG. 3A schematically illustrates plasmid pMON70811.

FIG. 3B schematically illustrates a derivative linear DNA molecule of pMON70811.

FIG. 3C schematically illustrates the insertion of 1mMON70811 into a host target site.

FIG. 4A schematically illustrates plasmid pMON70805.

FIG. 4B schematically illustrates a derivative linear DNA molecule of pMON70805.

FIG. 4C schematically illustrates the insertion of 1mMON70805 into a host target site.

FIG. 5 schematically illustrates a target recombination site in a genome for site-specific insertion of selected DNA.

FIGS. 6 and 7 schematically illustrate plasmids useful for expressing a recombinase encoding DNA.

FIG. 8A schematically illustrates plasmid pMON73562.

FIG. 8B schematically illustrates a derivative Agrobacterium T-DNA molecule of pMON73562.

FIG. 8C schematically illustrates the insertion of Agrobacterium T-DNA transferred from MON73562 into a host target site.

### DETAILED DESCRIPTION OF THE INVENTION

1. Definitions

Ancillary DNA means a DNA segment which is not one of the selected sequences desired for transformation into a recipient genome. Ancillary DNA may include bacterial origins of replication, plasmid sequences and marker genes, or any other sequences deemed necessary or optional for a effective transformation.

Circular DNA molecule means a circular, double-stranded or single-stranded DNA molecule which is useful for recombinase-mediated insertion of selected DNA into a specific recombination site in a target genome in an organism. A circular DNA molecule of this invention may contain three or more recombination sites, such as a lox site or frt site. Such a circular DNA molecule with at least three recombination sites serves as a site-directed molecule for insertion of selected DNA. Plasmids comprising at least three site-specific recombination sites are an example of a circular DNA molecule useful in the practice of the present invention.

Compatible Recombination Sites mean recombination sites which can recombine with each other to some degree under the influence of recombinase. For instance, Albert et al. (discussed above) reports that recombination between lox43 and lox44 sites is less efficient than between two loxP sites. Despite the lower efficiency of recombination lox43 and lox44 sites are considered compatible recombination sites.

Exogenous DNA refers to DNA which is not normally found next to the adjacent native DNA, i.e., a sequence not normally found in the host genome in an identical context. The DNA itself may be native to the host genome or may comprise the native sequence altered by the addition or deletion of one or more different regulatory elements or other sequences. The exogenous DNA may encode a protein or non-protein product, such as a tRNA, rRNA or an RNA effective for gene suppression. Likewise, “exogenous sequence” is a sequence of DNA not normally found in the host genome in an identical context. A transformation construct comprising a gene of interest and at least one recombination site, which originates or is produced outside of an organism, is an example of an exogenous DNA.

Expression means the combination of intracellular processes, including transcription and translation undergone by a coding DNA molecule such as a structural gene to produce a polypeptide or an RNA product.

Gene means a DNA sequence from which an RNA molecule is transcribed. The RNA may be an mRNA which encodes a protein product, an RNA which functions as an anti-sense molecule, or a structural RNA molecule such as a tRNA, rRNA or snRNA, or other RNA.

Genetic Transformation means a process of introducing a DNA sequence or construct (e.g., a vector or DNA construct in linear or circular form) into a cell or protoplast in which that exogenous DNA is incorporated into a chromosome or is capable of autonomous replication.

Linear DNA molecule means a linear, double-stranded or single-stranded DNA molecule which is useful for recombinase-mediated insertion of selected DNA into a specific recombination site in a target genome in an organism. A linear DNA molecule of this invention may contain one or more recombination sites, such as a lox site or a frt site. Such a linear DNA molecule with a recombination site serves as a site-directed molecule for insertion of selected DNA.

Non-Compatible Recombination Sites mean recombination sites which cannot recombine with each other under the influence of recombinase, or recombine with each other under the influence of a recombinase with a frequency of about 2% or less. See U.S. Pat. No. 6,465,254 (incorporated herein by reference) which discloses mutant lox sites comprising mutations in the core sequence of the lox site for which no CRE-dependent recombination was shown to occur.

Progeny means any subsequent generation, including the seeds and plants therefrom, which is derived from a particular parental plant or set of parental plants; the resultant progeny can be inbred or hybrid. Progeny of a transgenic plant of this invention can be, for example, self-crossed, crossed to a transgenic plant, crossed to a non-transgenic plant, and/or back-crossed.

Promoter means a recognition site on a DNA sequence or group of DNA sequences that provides an expression control element for a structural gene and to which RNA polymerase specifically binds and initiates RNA synthesis (transcription) of that gene.

R₀ Transgenic Plant means a plant which has been directly transformed with a selected exogenous DNA or has been regenerated from a cell or cell cluster which has been transformed with a selected exogenous DNA.

Regeneration means a process of growing a plant from a plant cell (e.g., plant protoplast, callus or explant).

Selected DNA means a DNA segment of nucleotide sequence which one desires to introduce into a plant genome by genetic transformation, e.g. a selected gene, a selected protein coding sequence, a selected antisense coding element, an expression regulatory element or a selected RNA interference (RNAi) coding element.

Selected Gene means a gene which one desires to have expressed or suppressed in a transgenic plant, plant cell or plant part. A selected gene can comprise regulatory elements, introns and protein coding elements or simply protein coding elements. A selected gene may be native or foreign to a host genome, but where the selected gene is present in the host genome, will include one or more regulatory or functional elements which differ from native copies of the gene. A selected gene may include, but is not limited to, genes imparting insect resistance, herbicide resistance, improved agronomic traits, improved quality traits or improved yield, and does not include ancillary sequences.

Site-directed insertion means insertion of an exogenous DNA into a target genome at a single recombination site via recombinase-mediated integration, such as a single lox site or a single frt site.

Site-specific recombination site means a DNA sequence that is recognized by and acted upon by an integrase type recombinase to effect DNA recombination, including but not limited to, an insertion, deletion or inversion of sequences directed by the recombination site sequences. Examples of recombinase and recombination site pairs described herein include, but are not limited to, FLP recombinase which catalyzes DNA recombination at frt recombination sites and CRE recombinase which catalyzes DNA recombination at lox recombination sites.

Stably transformed plant means a plant in which exogenous DNA is heritable. The exogenous DNA may be heritable as a fragment of DNA maintained in the plant cell and not inserted into the host genome. Preferably, the stably transformed plant comprises exogenous DNA inserted into the chromosomal DNA in the nucleus, mitochondria or chloroplast, most preferably in the nuclear chromosomal DNA.

Transformed cell means a cell in which the genomic DNA been altered by the insertion of an exogenous DNA molecule into the genome or by the cellular support of an autonomously replicating molecule comprising exogenous DNA.

Transgene means exogenous DNA which is intended to be, or has been, incorporated into a host genome or is capable of autonomous replication in a host cell and is capable of causing the expression of one or more cellular products. Transgenes may be directly introduced into a plant by genetic transformation, or may be inherited from a plant of any previous generation which was transformed with the exogenous DNA.

Transgenic plant means a plant or progeny plant of any subsequent generation derived therefrom, wherein the DNA of the plant or progeny thereof contains an introduced exogenous DNA segment not originally present in a non-transgenic plant of the same strain.

2. Recombination Systems

Site-specific recombinase/recombination site systems useful in this invention include, but are not limited to, the CRE/lox system of bacteriophage P1, the FLP/fit system of yeast, the Gin/gix system of phage Mu, and the R/RS system of the pSR1 plasmid from Xygosaccharomyces rouxii.

FLP recombinase has been employed with frt recombination sites to direct site-specific excision of parts of transgene DNA in maize and rice protoplasts by homologous recombination (U.S. Pat. No. 5,527,695, incorporated herein by reference). FLP/frt has also been used in stably transformed maize for site-directed excision of sequences inserted into the maize genome which are flanked by frt sites (U.S. Pat. Nos. 5,929,301 and 6,175,058 each of which is incorporated herein by reference). In the presence of FLP recombinase, the integration/excision reactions are reversible. It is possible, however, to sufficiently alter frt sites such that recombination occurs but is not reversible (U.S. Pat. No. 6,187,994, incorporated herein by reference), that is, favors a forward reaction relative to a reverse reaction.

CRE recombinase has been shown to mediate recombination between lox sites in yeast, plants such as tobacco and Arabidopsis (U.S. Pat. No. 5,658,772; Albert et al., 1995, each of which is incorporated herein by reference), as well as in mammalian cells such as mice (Sauer and Henderson, Proc. Natl. Acad. Sci. USA., 85(14):5166-5170, 1988; Fukushige and Sauer, Proc. Natl. Acad. Sci. USA, 89(17):7905-9, 1992; Sauer, Methods, 14:381-392, 1998, each of which is incorporated herein by reference). Site-specific integration of large BAC (bacterial artificial chromosome) fragments into plant and fungal genomes utilizing a CRE/lox recombination system has also been reported (Choi et al., Nuc. Acids Res., 28(7):e19, 2000, incorporated herein by reference). In addition, site-specific recombination in a plant plastid genome has been disclosed (PCT Publications WO 01/21768 and WO 01/29241, incorporated herein by reference).

CRE recombinase can contact and effect DNA recombination using a number of lox sites including, but not limited to, loxP (wild type; SEQ ID NO:1) and a number of variants of the wild type loxP site such as lox66 (Albert et al., Plant J., 7(4):649-659, 1995, incorporated herein by reference; SEQ ID NO:2). The DNA exchange directed by the lox sites occurs in the 8 bp spacer or “core” region and essentially effects an exchange of the 13 bp inverted repeats of the two lox sites involved. For example, site-directed recombination in which a single lox site on one DNA molecule recombines with a second single lox site on a second DNA molecule generates a sequence in which the integrated DNA is flanked by a lox site on either side. When the single lox sites on the separate molecules involved are identical, the two resultant lox sites adjacent to the inserted DNA are also identical. If, however, the two single lox sites on the starting molecules are not identical in sequence in the 13 bp inverted repeats, the two resultant lox sites adjacent to the inserted DNA will differ from the starting lox sites. For example, if a first single lox66 site (SEQ ID NO:2) is involved in site-directed integration with a second single lox71 site (SEQ ID NO:3), the resultant lox sites flanking the inserted DNA comprise sequences of loxP and lox72 sites (Albert et al., 1995; SEQ ID NO: 1 and SEQ ID NO:4, respectively).

Site-directed integration utilizing identical lox or frt sites on the two recombining molecules is a reaction that is easily reversed by the recombinase. To prevent the deletion of the inserted sequence, it is often desirable to remove the source of recombinase enzyme, for example, by segregation or by placing the recombinase gene under the control of an inducible promoter and removing the inducing source. Alternatively, one of skill in the art may use site-specific recombination sequences designed such that after the integration reaction, the resultant sites are non-compatible for a reverse reaction or recombine at a reduced rate (WIPO publication WO 01/11058, incorporated herein by reference) or such that two sites are “non-compatible” recombination substrates for the recombinase (U.S. Pat. No. 6,465,254, incorporated herein by reference).

Those skilled in the art will recognize that the integrase enzyme, such as CRE or FLP recombinase, can be provided to the target site or sites, such as lox or frt, by any means known in the art. For example, the recombinase can be transiently supplied by expression from a gene, and appropriate control sequences, that reside on a separately maintained plasmid within the host cells. The recombinase gene and appropriate control sequences can be inserted into the genome of the organism and stably expressed in the host cells. Alternatively, sexual crossing or breeding may be used to introduce the recombinase to cells containing the target lox or frt site or sites; in this instance, an organism such as plant containing the recombinase gene could be crossed to a plant containing the target lox or frt sites and progeny from this union would contain both the recombinase and the target site or sites. In some cases, mRNA coding for the desired recombinase can be introduced into the host cells to encode and provide the recombinase protein. In other cases, one may introduce isolated recombinase protein into a host cell comprising a target recombination site. In any of these cases, the promoter directing recombinase expression may be, but not limited to, constitutive or inducible in manner. One of skill in the art will also recognize that the genes for recombinase genes such as CRE or FLP may be isolated from bacteriophage P1 or Saccharomyces cerevisiae, respectively, and utilized directly in a new host system or the gene sequence may be optimized for codon usage for expression in the transgenic host. In a similar fashion, one of skill in the art will recognize that naturally occurring as well as synthetic target sites may be recognized and mediate recombination with an appropriate recombinase.

3. Recombination Site-Containing Genome for Transformation

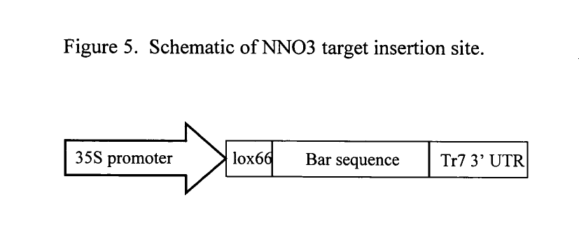

Linear or circular DNA molecules of this invention comprise a site-specific recombination site for integration into a compatible target site-specific recombination site in a host genome. In one embodiment, a target site in the genome comprises a specific recombination site adjacent to a 5′ regulatory sequence, that is a promoter, and a 3′ untranslated region, that is sequence for the completion of transcription (see for example, FIG. 5).

A number of suitable promoters that are active in plant cells are known to those of skill in the art and comprise constitutive, tissue specific, inducible, developmentally regulated, and the like. See the background section of U.S. Pat. No. 6,437,217 (incorporated herein by reference in its entirety) for a description of a wide variety of promoters that are functional in plants. A 35S promoter from Cauliflower Mosaic Virus (CaMV) or a rice actin 1 intron 1 promoter are useful for the practice of this invention. It is preferable that the particular promoter selected result in the production of an effective amount of RNA and/or protein encoded by a selected DNA in a transgenic plant. Other sequences necessary for gene expression can be included such as transit peptides, signal peptides, enhancers, and the like. In another embodiment, a target site comprises a specific recombination site. Target sites can be created in the genome using any genetic transformation method suitable for incorporating or adding exogenous DNA to a genome, including but not limited to, microprojectile bombardment and Agrobacterium-mediated transformation methods. Preferred site-specific recombination sites include lox and frt sites, most preferably a lox site.

4. DNA Molecules for Transformation

The linear or circular DNA molecules of the present invention are made using molecular biology methods known to those of ordinary skill in the art.

The linear DNA molecules of this invention comprising selected DNA and at least one recombination site can be synthesized from single-stranded or double-stranded DNA. Linear DNA molecules may be generated from a variety of DNA source molecules including, but not limited to, circular double-stranded or single-stranded plasmids with or without ancillary sequences, or RNA or RNA which has been reverse-transcribed into complementary DNA (cDNA) in any of the previously described forms. Those skilled in the art can use standard molecular biology techniques to modify source RNA or DNA molecules to construct a starting template which contains only the sequences of interest. In this regard, those skilled in the art will recognize that RNA molecules are typically reverse-transcribed into complementary DNA for use with standard molecular biology techniques. Thus, DNA source molecules used in the practice of this invention may have been derived from RNA sources.

Site-directed linear DNA molecules can be synthesized from double-stranded, circular DNA molecules containing the selected DNA, a recombination site DNA and optional ancillary sequences.

If the DNA source material is a circular double-stranded DNA molecule containing ancillary DNA, it may be necessary to remove part or all of the ancillary DNA. Amplification methods such as polymerase chain reaction (PCR) may be used to produce linear or circular molecules lacking ancillary sequences. Primers may be designed such that a desired recombination site may be included in the amplified product. Alternatively, restriction enzymes may be used to digest the starting circular plasmid to remove the ancillary sequences as one or more fragments of DNA while retaining the DNA of interest, as well as site-directed recombination sequences, on a single contiguous DNA fragment. The various digestion products are separated, for example on an agarose gel, and the fragment(s) of interest isolated away from the ancillary sequences. The linear fragment(s) with the DNA sequences of interest can be further purified if needed and used in transformation.

If the DNA source material is linear double-stranded DNA containing ancillary DNA sequence, it may be desirable to remove the ancillary DNA sequences. Amplification methods such as PCR may be used to produce linear DNA molecules lacking ancillary DNA sequences. Primers may be designed such that a desired recombination site may be included in the amplified product. Alternatively, restriction enzymes may be used to digest the starting linear double-stranded DNA to remove the ancillary DNA sequences as one or more fragments of DNA while retaining the DNA sequences of interest in one or more DNA fragments. The various digestion products can be separated, for example on an agarose gel, and the fragment or fragments of interest isolated away from the ancillary DNA sequences. The fragment with the DNA sequences of interest can be further purified if needed and used in transformation.

Those skilled in the art can also prepare linear DNA molecules for transformation from two or more fragments containing the DNA sequences and recombination site sequences of interest. If the restriction site DNA is not compatible for ligation, it can be modified, e.g. by preparing blunt ends on the fragment or adding linkers with compatible ends or introducing a restriction site, to facilitate creating a linear DNA molecule by ligation. Other standard molecular biology methods can be used to remove ancillary sequences and reform the site-directed linear DNA molecules.

In one aspect of the invention, a linear DNA molecule can be inserted at a recombination site in a genome located downstream from a promoter so that after insertion the linear DNA molecule will be operably linked to a promoter which is native to the organism. In other aspects of the invention, a linear DNA molecule may contain an expression unit of DNA comprising a promoter operably linked to a gene of interest further linked to a 3′ untranslated region (3′UTR, also known as a 3′ end or simply 3′) and other sequences necessary for desired expression.

It is preferred that the particular promoter selected should be capable of causing sufficient expression to result in the production of an effective amount of RNA and/or protein product encoded by the exogenous DNA of interest.

A site-directed linear DNA molecule may contain one or more site-specific recombination sites. For purposes of illustrating the invention, reference will be made to the CRE/lox recombination system with the understanding that other recombination systems are within the scope of this invention. For example, lox recombination sites allow CRE-recombinase-mediated integration of a linear DNA molecule into the host DNA, preferably into a target lox site in the nuclear chromosomal DNA and most preferably, in a location comprising a lox66 (SEQ ID NO:2) or a lox71 site (SEQ ID NO:3). The inclusion of a first lox recombination site in a linear DNA molecule can result in the targeted insertion of the entire molecule into the nucleic acid comprising a second single target lox site. The inclusion of a first and a second lox site in a site-directed linear molecule can result in the targeted insertion of an entire linear DNA molecule into the nucleic acid comprising a third single target lox site. The inclusion of a first, second and a third lox site in a DNA molecule can result in the targeted insertion of the entire molecule into the target nucleic acid comprising a fourth single target lox site. The use of a single lox site in the target nucleic acid molecule is thought to reduce or eliminate extraneous recombination events such as deletion, inversion or duplication of a region that may occur when sequences are flanked by lox or other homologous sequences.

Location of the lox site or sites on a linear DNA molecule may vary. A molecule containing a first single lox site may contain the sequence interiorly on the molecule, or, preferably, at or near either the 5′ or 3′ end of the molecule, most preferably at the 5′ end. A linear DNA molecule containing first and second lox sites may contain the sequences interiorly on the molecule or, preferably, the lox sites are located, one each, at or near the 5′ and 3′ ends of the molecule. The lox sites may or may not be identical in sequence. Preferably the sequences of multiple sites on a linear DNA molecule are not identical in sequence. Linear DNA molecules containing first, second and third lox sites may contain the sequences interiorly on the molecule or, preferably, a first lox site may be located interiorly and the second and third lox sites may be located at or near the 5′ and 3′ ends of the molecules. The lox sites may or may not be identical in sequence. In a preferred embodiment, the second and third lox sites are identical in sequence and the first, interiorly located lox site is of a different sequence and is essentially non-compatible with the second and third lox sites.

In a similar fashion, circular a DNA molecule comprising three or more recombination sites can be constructed and propagated by tools known to those of skill in the art of molecular biology. As with the preparation of linear DNA molecules for use with the present invention, sequences in the circular molecules may be derived from RNA, DNA, cDNA, single or double stranded DNA sources, and various fragments of DNA may be joined together to form the desired circular molecule. The DNA molecules are joined by tools known to the skilled molecular biologist and include ligation, DNA amplification and the like. Propagation of the circular DNA molecules in host cells such as bacterial cells, e.g., E. coli or Agrobacterium, is known to those of skill in the art. Circular DNA molecules comprising three or more recombination sites may also be prepared using DNA amplification methods such as, but not limited to, polymerase chain reaction. A discussion of the preparation of circular molecules using in vitro techniques, particularly the preparation of circular DNA molecules lacking ancillary sequences, may be found in U.S. Patent Application 20030100077, incorporated herein by reference in its entirety. Circular molecules for use with present invention are designed such that the recombination sites may flank ancillary sequences for removal during or post transformation processes. Site specific recombination sites include but are not limited to lox and frt sites, preferably lox sites.

Commonly used methods of plant transformation, including biolistic transformation as well as Agrobacterium-mediated transformation, result in the introduction of DNA into the target genome at random locations, i.e. at a non-specific location, in the genome of a parental maize line. An advantage of the current invention are methods useful to target exogenous DNA insertion in order to achieve site specific integration, e.g. to target an exogenous DNA into the genome at a predetermined site from which it is known that gene expression occurs. Several site specific recombination systems exist which are known to function in plants include Cre-lox as disclosed in U.S. Pat. No. 4,959,317 and FLP-FRT as disclosed in U.S. Pat. No. 5,527,695, both incorporated herein by reference.

5. Transformation Constructs

Transformation constructs for site-specific integration using DNA molecules of this invention can be prepared by those skilled in the art using any of a variety of DNA construction or fabrication techniques including but not limited to DNA amplification methods, cloning methods including traditional ligation methods as well as integrase methods (e.g., GATEWAY™ cloning technology (available from Invitrogen Life Technologies, Carlsbad, Calif.), and other molecular biology methods.

The aspects of this invention are thus not limited to any particular DNA sequences. One important use of the DNA molecules of this invention is to create transgenic cells or organisms which express a selected DNA which encodes a particular protein or RNA product. Useful exogenous DNA sequences to include in the site-directed DNA molecules of the invention are exemplified by sequence encoding proteins, polypeptide products, RNA molecules, marker genes, or combinations thereof. It also is contemplated that expression of one or more genes may be suppressed upon induction of a promoter operably linked to a selected DNA.

In certain embodiments, the present inventors contemplate the transformation of a recipient cell with more than one transformation construct, a co-transformation. Preferred components likely to be included with vectors used in the current invention are as follows: regulatory elements including 3′ untranslated regions, 5′ untranslated regions, enhancers, introns, signal peptide coding sequences, transit peptide coding sequences, selectable marker genes, screenable marker genes, and the like. The rice actin 1 promoter with a rice actin intron is especially useful in the practice of the present invention. A discussion of useful plant transformation constructs which can be prepared by those of ordinary skill in the art can be found, for example, in U.S. Pat. No. 6,437,217 which discloses a maize RS81 promoter and U.S. Pat. No. 5,641,876 which discloses a rice actin promoter, each of which is incorporate herein by reference.

Molecules used for site-specific integration of plant cells will, of course, generally comprise at least the cDNA, often the gene or genes including introns, which one desires to introduced into and have expressed in the host cells. These DNA molecules can include sequences such as promoters, enhancers, 3′ untranslated regions, polylinkers, or even regulatory genes as desired. The DNA molecules chosen for cellular introduction may encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes. Preferred components likely to be included with the linear or circular DNA molecules used in the current invention are as follows.

It also is contemplated that expression of one or more genes may be suppressed upon induction of a promoter operably linked to a selected DNA. For instance, when suppression of protein expression is the intended objective, the selected DNA can be designed to produce a gene silencing effect, e.g. by an antisense or RNAi mechanism. Gene suppression means any of the well-known methods for suppressing an RNA transcript or production of protein translated from an RNA transcript, including post-transcriptional gene suppression and transcriptional suppression. Post-transcriptional gene suppression is mediated by double-stranded RNA having homology to a gene targeted for suppression. Gene suppression by RNA transcribed from an exogenous DNA construct comprising an inverted repeat of at least part of a transcription unit is a common feature of gene suppression methods known as anti-sense suppression, co-suppression and RNA interference. Transcriptional suppression can be mediated by a transcribed double-stranded RNA having homology to promoter DNA sequence to effect what is called promoter trans-suppression. More particularly, post transcriptional gene suppression by inserting an exogenous DNA construct with anti-sense oriented DNA to regulate gene expression in plant cells is disclosed in U.S. Pat. Nos. 5,107,065 and U.S. Pat. No. 5,759,829, each of which is incorporated herein by reference in its entirety. Transgenic plants transformed using such anti-sense oriented DNA constructs for gene suppression can comprise DNA arranged as an inverted repeat, as disclosed by Redenbaugh et al. in “Safety Assessment of Genetically Engineered Flavr Savr™ Tomato, CRC Press, Inc. (1992). Inverted repeat insertions can comprises a part or all of a T-DNA construct. The DNA molecules of this invention may be used for targeted, site-directed integration into a genome to effect gene suppression.

The inventors also contemplate that, where both an expressible gene that is not necessarily a marker gene is employed in combination with a marker gene, one may employ the separate genes on either the same or different DNA molecules for transformation. In the latter case, the different DNA molecules can be delivered concurrently to different recombination sites on a target genome to maximize cotransformation.

6. Selected DNA for Transgenic Plants

This invention provides linear DNA molecules containing selected exogenous DNA and at least one recombination site, or circular DNA molecules DNA molecules containing selected exogenous DNA and at least three recombination sites, for site-directed integration into a compatible recombination site in a plant and subsequent expression of the selected exogenous DNA in the plant. Use of preselected, characterized recombination sites for integration of the selected DNA will allow reproducible expression of transformed sequences.

The choice of a selected DNA for expression in a plant host cell in accordance with the invention will depend on the purpose of the transformation. One of the major purposes of transformation of crop plants is to express any gene which one desires to have expressed for imparting a commercially desirable, agronomically important or end-product traits to the plant. Such traits include, but are not limited to, herbicide resistance, herbicide tolerance, insect resistance, insect tolerance, disease resistance, disease tolerance (viral, bacterial, fungal, nematode), stress tolerance, stress resistance, as exemplified by resistance or tolerance to water-deficit, heat, chilling, freezing, excessive moisture, salt stress and oxidative stress, increased yield, food content and value, increased feed content and value, physical appearance, male sterility, female sterility, drydown, standability, prolificacy, starch quantity and quality, oil quantity and quality, protein quality and quantity, amino acid composition, and the like. It is also anticipated that expression of exogenous DNA encoding antisense RNAs or other RNA molecules are included as useful means for modifying plant phenotype.

In certain embodiments of the invention, transformation of a recipient cell may be carried out with more than one selected DNA. Two or more exogenous coding sequences also can be supplied in a single transformation event using either distinct DNA molecules, or using a single DNA molecule incorporating two or more exogenous DNA sequences.

7. Transgene Detection and Assays of Transgene Expression

To confirm the presence of the exogenous DNA in regenerating plants, a variety of assays may be performed. Such assays include, for example, molecular biological assays such as Southern and Northern blotting and PCR; biochemical assays such as detecting the presence of a protein product, e.g., by immunological means (ELISAs and Western blots) or by enzymatic function; plant part assays such as leaf or root assays; and in some cases phenotype analysis of a whole regenerated plant. Such assays are known to those of skill in the art.

To more precisely characterize the presence of transgenic material in a transformed plant, one skilled in the art can identify the point of insertion of the transgene and, using the sequence of the recipient genome flanking the transgene, develop an assay that specifically and uniquely identifies a particular insertion event (see for example PCT Publication WO 02057471, incorporated herein by reference in its entirety). Preferred means for determining the presence of a transgene in a transformed plant include the use of Southern blotting and TaqMan® genomic assays (available from Applied Biosystems, Foster City, Calif.).

Assays may also be employed to determine the relative efficiency of transgene expression. For plants, expression assays may comprise a system utilizing embryogenic or non-embryogenic cells, or alternatively, whole plants. An advantage of using cellular assays is that regeneration of large numbers of plants is not required. However, the systems are limited in that promoter activity in the non-regenerated cells may not directly correlate with expression in a plant. Additionally, assays of tissue or developmental specific promoters are generally not feasible. The biological sample to be assayed may comprise nucleic acid molecules isolated from the cells of any plant material according to standard methodologies well known to those of ordinary skill in the art. The nucleic acid molecules may be genomic DNA, or fractionated or whole cell RNA. Where RNA is used, it may be desired to convert the RNA to a complementary DNA (cDNA). In one embodiment of the invention, the RNA is whole cell RNA; in another, it is poly-adenylated RNA. Normally, the nucleic acid is amplified. Assay techniques include, but are not limited to, fluorescent in situ hybridization (FISH), direct DNA sequencing, pulsed field gel electrophoresis (PFGE) analysis, Southern or Northern blotting, single-stranded conformation analysis (SSCA), RNase protection assay, allele-specific oligonucleotide (ASO), dot blot analysis, denaturing gradient gel electrophoresis, PCR, RT-PCR, quantitative RT-PCR, RFLP and PCR-SSCP. Preferred means for detecting the expression of a transgene include the use of selectable markers, protein assays, Westerns, Northerns, RT-PCR, quantitative RT-PCR, and TaqMan® genomic assays.

8. Methods for Plant Transformation

There are a variety of suitable methods for plant transformation for use with the linear or circular DNA molecules of this invention and include virtually any method by which DNA can be introduced into a cell, such as by direct delivery of DNA such as by PEG-mediated transformation of protoplasts, by electroporation, by agitation with silicon carbide fibers, by acceleration of DNA coated particles, and by transfer of single-stranded transfer-DNA (T-DNA) via Agrobacterium-mediated transformation. Typically, an Agrobacterium vector comprises at least 2 border sequences, a left border and a right border. During Agrobacterium infection and transformation of the host, the DNA between the two border sequences is transferred to the plant via a single-stranded T-DNA molecule for integration into the host genome.

Through the application of techniques such as these, maize cells, as well as those of virtually any other plant species, may be stably transformed, and these cells developed into transgenic plants. In certain embodiments, acceleration methods are preferred and include, for example, microprojectile bombardment and the like. Useful methods of plant transformation are microprojectile bombardment as illustrated, for example, in U.S. Pat. Nos. 5,015,580; 5,550,318; 5,538,880; 6,160,208; 6,399,861 and 6,403,865 and Agrobacterium-mediated transformation as illustrated, for example, in U.S. Pat. Nos. 5,635,055; 5,824,877; 5,591,616; 5,981,840 and 6,384,301, all of which are incorporated herein by reference.

9. Recipient Cells for Transformation

Transformation methods of this invention to provide plants in which a linear or circular DNA molecule is integrated into a target genome at a site-specific recombination site are preferably practiced in tissue culture on media and in a controlled environment. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Those cells which are capable of proliferating as callus also are recipient cells for genetic transformation. The present invention provides techniques for transforming immature embryos and subsequent regeneration of fertile transgenic plants. Practical transformation methods and materials for making transgenic plants of this invention, e.g. various media and recipient target cells, transformation of immature embryos and subsequent regeneration of fertile transgenic plants are disclosed, for example, in U.S. Pat. No. 6,194,636 and U.S. patent application Ser. No. 09/757,089, which are incorporated herein by reference.

10. Production and Characterization of Stably Transformed Plants

After effecting transformation and site-directed integration of exogenous DNA to recipient cells, the next steps generally concern identifying the transformed cells for further culturing and plant regeneration. As mentioned herein, in order to improve the ability to identify transformants, one may desire to employ a selectable or screenable marker gene as, or in addition to, the expressible gene of interest. In this case, one would then generally assay the potentially transformed cell population by exposing the cells to a selective agent or agents, or one would screen the cells for the desired marker gene trait.

It is believed that DNA is introduced into only a small percentage of target cells in any one experiment. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include those conferring resistance to antibiotics such as kanamycin (nptII), hygromycin B (aph IV) and gentamycin (aac3 and aacC4) or resistance to herbicides such as glufosinate (bar or pat) and glyphosate (EPSPS). Examples of such selectable markers are illustrated in U.S. Pat. Nos. 5,550,318; 5,633,435; 5,780,708 and 6,118,047, all of which are incorporated herein by reference.

Cells that survive the exposure to the selective agent, or cells that have been scored positive in a screening assay, may be cultured in media that supports regeneration of plants. Ideally, seed from the transgenic plant is collected. Screenable markers which provide an ability to visually identify transformants can also be employed, e.g., a gene expressing a colored or fluorescent protein such as a luciferase or green fluorescent protein (GFP) or a gene expressing a beta-glucuronidase or uidA gene (GUS) for which various chromogenic substrates are known.

11. Breeding Plants of the Invention

This invention contemplates both plants directly regenerated from cells which have been transformed with an exogenous DNA construct of this invention as well as progeny of such plants, e.g. inbred progeny and hybrid progeny of transformed plants. This invention contemplates transgenic plants produced by direct transformation with an exogenous DNA construct of this invention and transgenic plants made by crossing a plant having a construct of the invention to a second plant lacking the construct. Crossing can comprise the following steps:

(a) plant seeds of the first parent plant (e.g. non-transgenic or transgenic) and a second parent plant having a transgenic exogenous DNA construct;

(b) grow the seeds of the first and second parent plants into plants that bear flowers;

(c) pollinate a flower from the first parent plant with pollen from the second parent plant; and

(d) harvest seeds produced on the parent plant bearing the fertilized flower.

It is often desirable to introgress transgenes into elite varieties, e.g. by backcrossing, to transfer a specific desirable trait from one source to an inbred or other plant that lacks that trait. This can be accomplished, for example, by first crossing a superior inbred (A) (recurrent parent) to a donor inbred (non-recurrent parent), which carries the appropriate gene(s) for the trait in question, for example, a construct prepared in accordance with the current invention. The progeny of this cross first are selected in the resultant progeny for the desired trait to be transferred from the non-recurrent parent, then the selected progeny are mated back to the superior recurrent parent (A). After five or more backcross generations with selection for the desired trait, the progeny are hemizygous for loci controlling the characteristic being transferred, but are like the superior parent for most or almost all other genes. The last backcross generation would be selfed to give progeny which are pure breeding for the gene(s) being transferred, i.e. one or more transformation events.

Therefore, through a series of breeding manipulations, a selected transgene may be moved from one line into an entirely different line without the need for further recombinant manipulation. Transgenes are valuable in that they typically behave genetically as any other gene and can be manipulated by breeding techniques in a manner identical to any other corn gene. Therefore, one may produce inbred plants which are true breeding for one or more transgenes. By crossing different inbred plants, one may produce a large number of different hybrids with different combinations of transgenes. In this way, plants may be produced which have the desirable agronomic properties frequently associated with hybrids (“hybrid vigor”), as well as the desirable characteristics imparted by one or more transgene(s).

Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized. The usefulness of marker assisted selection in breeding transgenic plants of the current invention, as well as types of useful molecular markers, such as but not limited to SSRs and SNPs, are discussed in PCT Application WO/02062129 and U.S. Patent Application Nos. 2002133852, 20030049612, and 2003005491 each of which is incorporated herein by reference in their entirety.

The ultimate goal in plant transformation is to produce plants which are useful to people. In this respect, transgenic plants created in accordance with the current invention may be used for virtually any purpose deemed of value to the grower or to the consumer. For example, one may wish to harvest seed for planting purposes, or products may be made from the seed itself such as oil, starch, animal or human food, pharmaceuticals, and various industrial products. Maize is used extensively in the food and feed industries, as well as in industrial applications. Further discussion of the uses of maize can be found, for example, in U.S. Pat. Nos. 6,194,636; 6,207,879; 6,232,526; 6,426,446; 6,429,357; 6,433,252, 6,437,217; 6,515,201 and 6,583,338; and PCT Publication WO 02/057471, each of which is specifically incorporated herein by reference in its entirety.

### 12. EXAMPLES

The following examples are included to illustrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

Preparation of DNA Molecules for Transformation

A) DNA Constructs

One of skill in the art will recognize from the descriptions herein that a wide variety of linear or circular molecules can be prepared and used for transformation in accordance with the instant invention. For illustrative purposes, examples of particular DNA molecules containing one, two or three lox sites are described. Plasmids used to maintain the desired sequences as circular DNA molecules are designated as pMON### while the linear molecules isolated from the plasmid molecules are designated as 1mMON###.

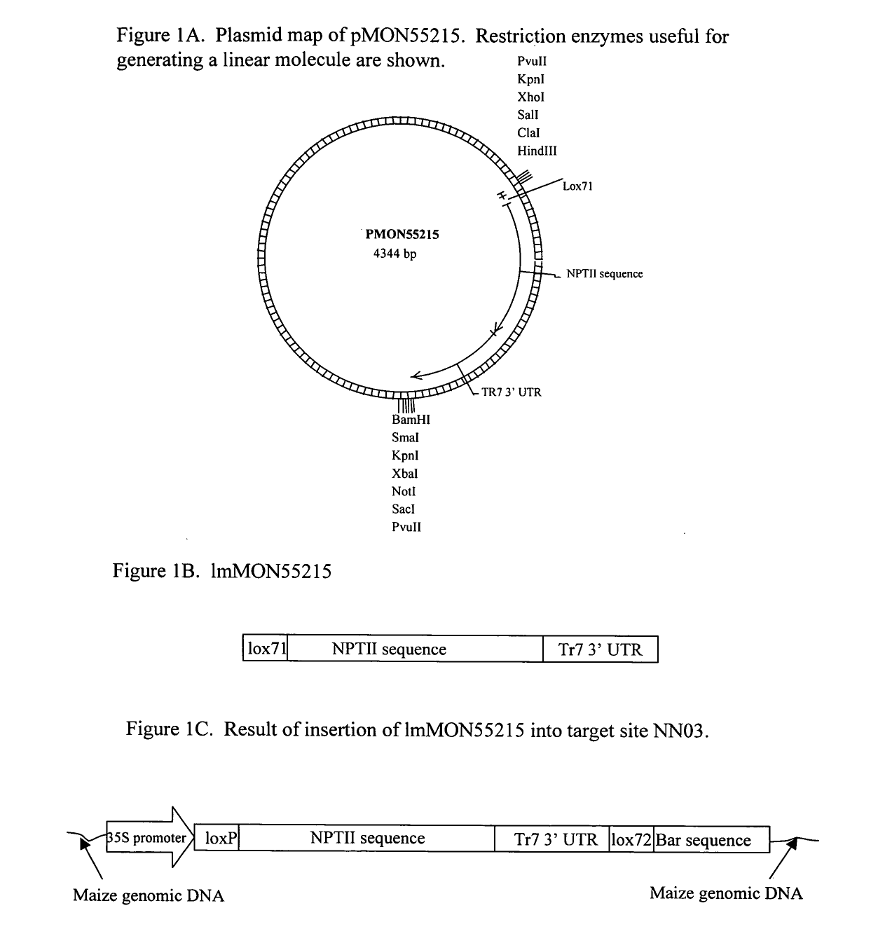

One lox site: A useful linear DNA molecule for targeted integration comprising a single lox71 site (SEQ ID NO:3), a promoterless neomycin phosphotransferase II selectable marker gene (NPT II) and a T7 3′UTR is 1mMON55215. The plasmid used for the maintenance of these sequences and for use as a circular DNA is pMON55215 (see FIG. 1A).

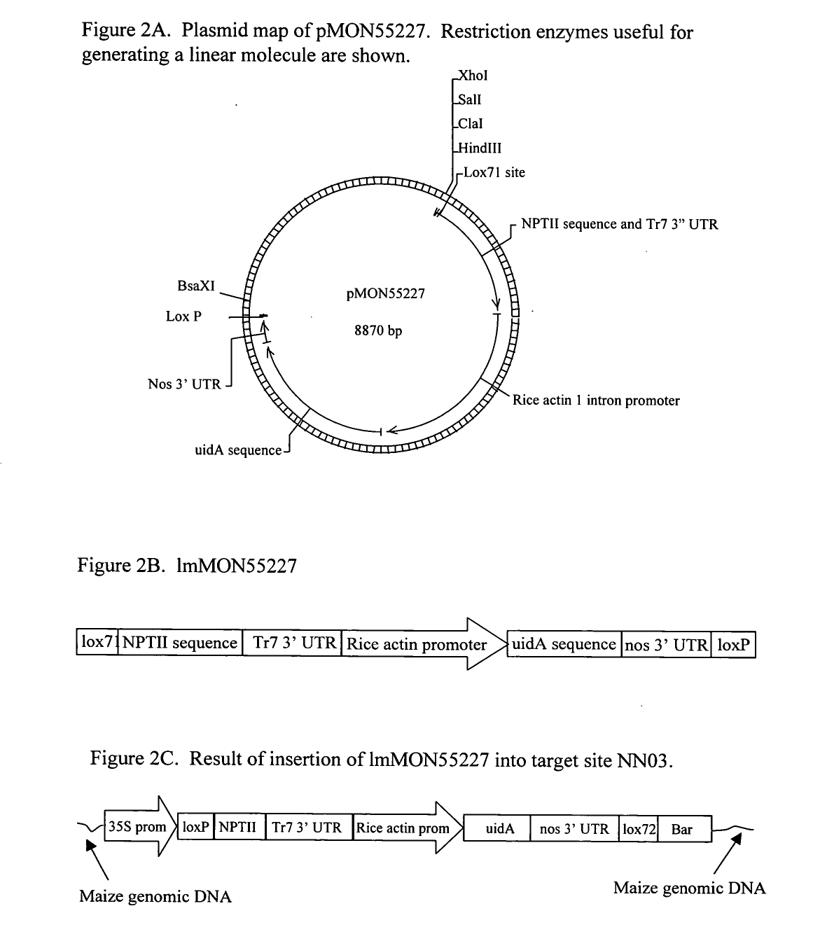

Two lox sites: A useful linear DNA molecule for targeted integration comprising a first single loxP site (SEQ ID NO: 1; 34 bp), a promoterless NPT II selectable marker gene, a T7 3′UTR, a rice actin 1 intron 1 promoter operably linked to a P-glucuronidase screenable marker gene (uidA gene; the protein product is commonly referred to as GUS), a nos 3′UTR and a second single lox71 site (SEQ ID NO:3) is 1mMON55227. The plasmid used for the maintenance of these sequences and for use as a circular DNA is pMON55227 (see FIG. 2A).

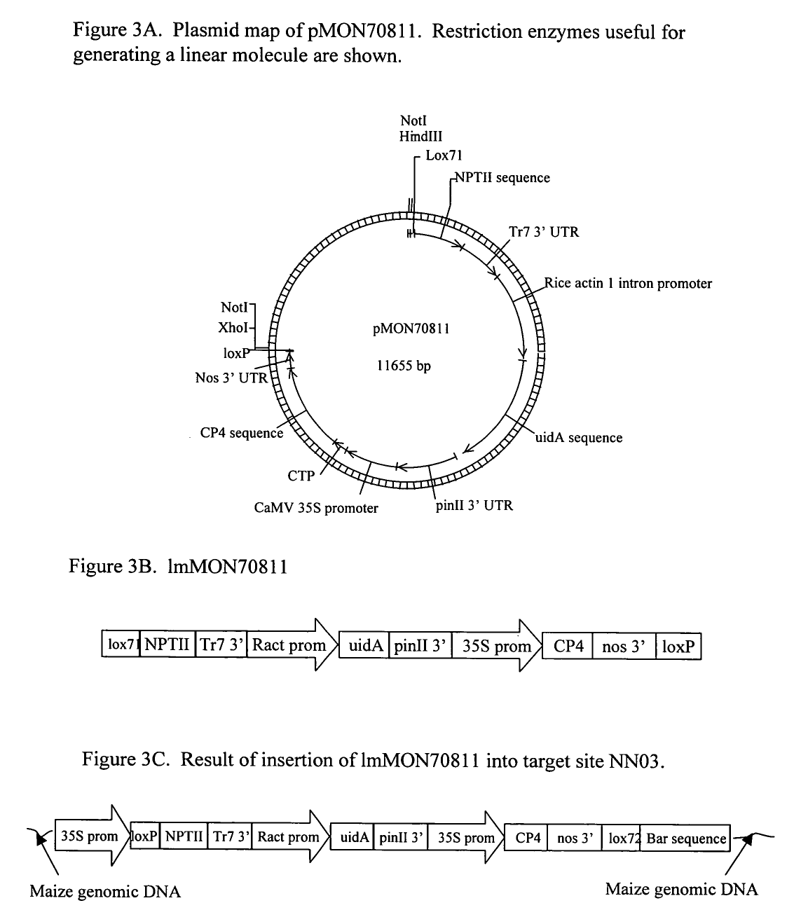

A second linear DNA molecule useful for targeted integration comprising a first single lox71 site (SEQ ID NO:3), a promoterless NPT II selectable marker gene, a T7 3′UTR, a rice actin 1 intron 1 promoter operably linked to a P-glucuronidase screenable marker gene (uidA gene; the protein product is commonly referred to as GUS), a nos 3′UTR and a second single loxP site (SEQ ID NO: 1) is 1mMON70811. The plasmid used for the maintenance of these sequences and for use as a circular DNA is pMON70811 (see FIG. 3A).

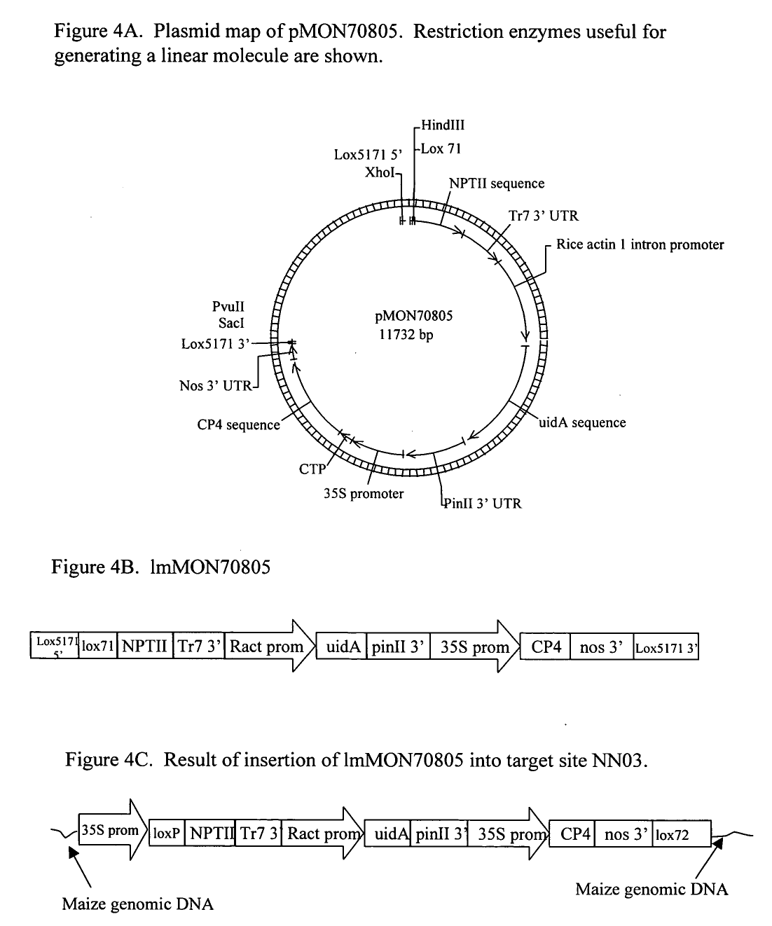

Three lox sites: A useful linear DNA molecule for targeted integration comprising a first single lox5171 5′ site (SEQ ID NO:5), a second single lox71 site (SEQ ID NO:3), a promoterless NPT II selectable marker gene, a T7 3′UTR, a rice actin 1 intron 1 promoter operably linked to a β-glucuronidase screenable marker gene (uidA gene; the protein product is commonly referred to as GUS), a pinII 3′UTR, and a glyphosate resistant EPSPS gene (CP4; U.S. Pat. No. 5,627,061 incorporated herein by reference) operably linked to a 35S promoter, an Arabidopsis EPSPS transit peptide, a nos 3′UTR and a third single lox5171 3′ site (SEQ ID NO:6) is MON70805. The plasmid used for the maintenance of these sequences and use as a circular DNA is pMON70805 (see FIG. 4A).

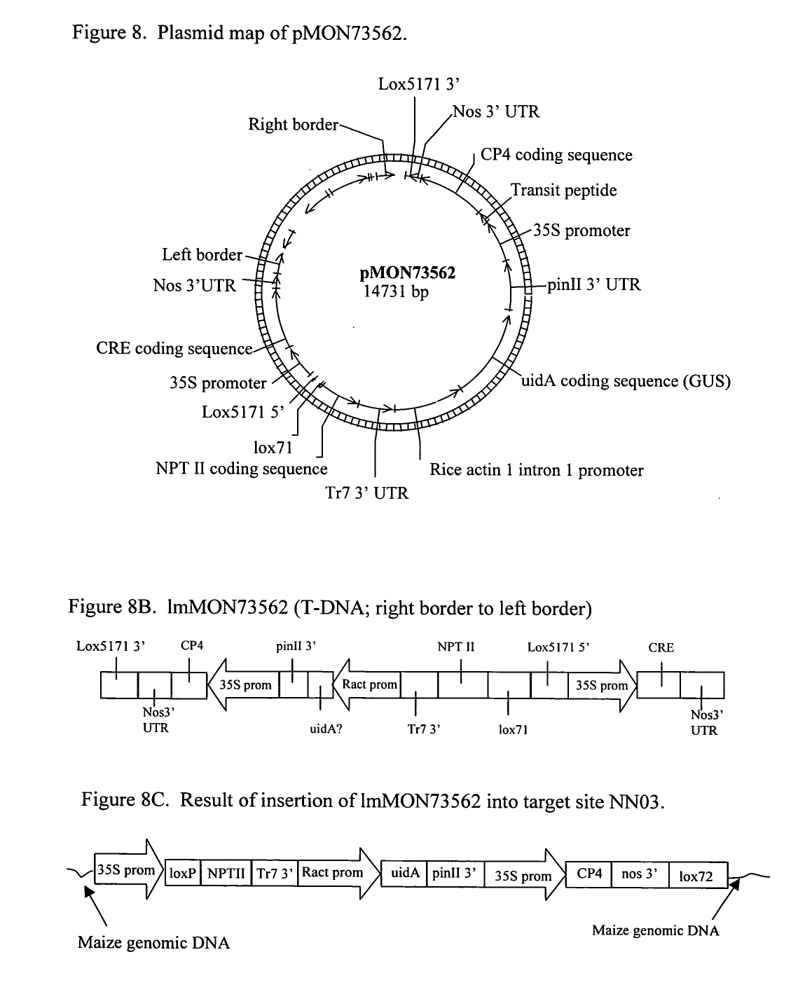

Another molecule with three lox sites useful for targeted integration studies comprising a left border, a nos 3′ UTR, a CRE recombinase gene operably linked to a CaMV 35S promoter, a lox5171 5′ site, a lox71 site, a promoterless NPT II selectable marker gene, a T7 3′ UTR, a rice actin 1 intron 1 promoter operably linked to a β-glucuronidase screenable marker gene (uidA gene; the protein product is commonly referred to as GUS), a pinII 3′ UTR, a CaMV 35S promoter operably linked to a chloroplast transit peptide and a glyphosate resistant EPSPS gene (CP4; U.S. Pat. No. 5,627,061 incorporated herein by reference), a nos 3′ UTR, a lox5171 3′ site and a right border is pMON73562 (see FIG. 8A).

B. Preparation of Linear and Plasmid DNA Molecules

Linear DNA molecules were prepared for transformation. Plasmid vectors pMON55215, pMON55227, pMON70805 or pMON70811 were restriction enzyme-digested to liberate a DNA fragment comprising one, two or three lox sites and selected exogenous DNA. The digestion products were dephosphorylated, preferably using calf alkaline intestinal phosphatase (Roche Molecular Biochemicals, Indianapolis, Ind.), and purified using a QIAQuick Nucleotide cleanup kit according to manufacturer's instructions (QIAGEN Inc., Valencia, Calif.). The ends of the molecule were then made blunt by using Klenow enzyme in the presence of all four nucleotides and conditions as recommended by the manufacturer (Roche Molecular Biochemicals, Indianapolis, Ind.). The products were separated on an agarose gel and the fragment containing the selected sequences was isolated. The DNA was purified away from the agarose support using QIAquick Gel Elution kit (QIAGEN Inc., Valencia, Calif.). The eluted DNA was quantitated and subsequently used for transformation. The linear molecules derived from the plasmid vectors pMON55215 (one lox site), pMON55227 or pMON70811 (two lox sites) or pMON70805 (three lox sites), designated as ImMON55215 (FIG. 1B), ImMON55227 (FIG. 2B), ImMON70811 (FIG. 3B) or 1mMON70805 (FIG. 4B), respectively, were used for particle bombardment transformation.

Circular plasmid DNA molecules comprised pMON55215, pMON55227, pMON70811 and pMON70805, each of which contained ancillary sequences in addition to the selected DNAs of interest. Vector pMON73562 was also used as a circular molecule for studying targeted site directed integration when the DNA was delivered by Agrobacterium-mediated transformation (FIG. 8B). These molecules were capable of replication in a bacterial host and were grown, isolated, purified and quantitated using standard molecular biology techniques familiar to one of skill in the art.

Purified DNA molecules, derived either from circular or linear molecule sources, were used in particle bombardment transformation experiments except for pMON73562 which was administered to the recipient cells via Agrobacterium-mediated transformation. All linear or plasmid molecules used in the transformation experiments contained a first single lox site (MON55215), first and second single lox sites (MON55227 or MON70811) or first, second and third single lox sites (MON70805 pr MON73562). To effect site-directed integration into a host genome, it was necessary to provide a single target lox site in a maize genome as well as CRE-recombinase.

The method of site directed integration using linear molecules of the instant invention was carried out using maize line H99. The site-specific target site in the maize background comprised, from 5′ to 3′, a 35S promoter, a lox66 site (SEQ ID NO:2), a bar selectable marker gene (U.S. Pat. No. 5,550,318 incorporated herein by reference) and a T7 3′UTR sequence. This target site is referred to as NN03 (see FIG. 5). In this case, site-directed insertion of a linear molecule into the lox66 NN03 site operably linked the selected DNA to the 35S promoter.

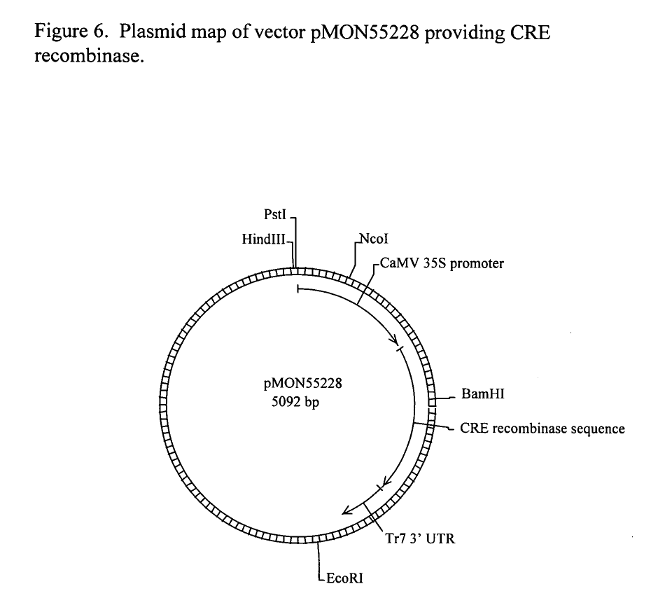

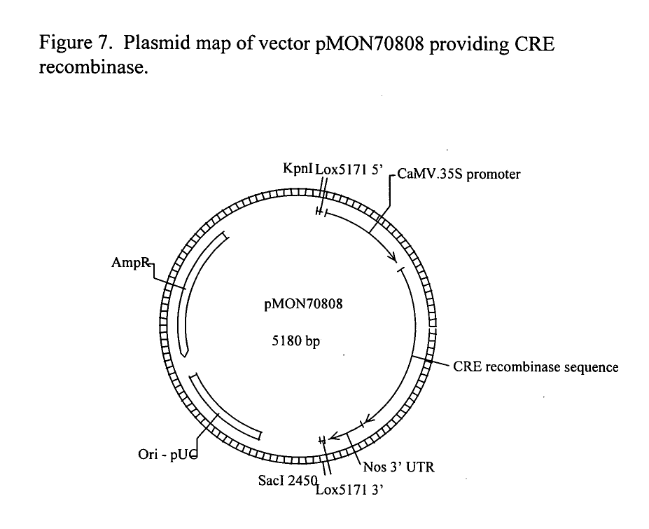

CRE-recombinase was provided to the linear or circular double-stranded DNA molecules from one of two plasmid vectors and could be provided either as a plasmid molecule or as a separate linear molecule to the host cells. pMON55228 (FIG. 6) comprised a CRE coding sequence operably linked to a 35S promoter and a Tr7 3′UTR. PMON70808 comprised a first single lox5171 3′ site (SEQ ID NO:6), CRE coding sequence operably linked to a 35S promoter, a nos 3′ UTR and a second single lox5171 5′ site (SEQ ID NO:5).

The CRE recombinase coding DNA was isolated from pMON55228 or pMON70808 by digestion of the plasmid with restriction enzymes. The digestion products were separated on agarose gels and the fragment containing the promoter, CRE recombinase coding sequence and 3′ untranslated region was isolated using standard molecular techniques. For co-bombardment transformation, approximately 5000 to 5 ng, preferably 3000 to 5 ng, preferably 1500 to 5 ng, preferably 1000 to 5 ng, preferably 500 to 5 ng, or preferably 250 to 5 ng, or most preferably 100 to 5 ng of linear molecules or circular molecules were mixed with approximately 5 to 50 ng of pMON55228 or pMON70808. It is contemplated that pMON55228 or pMON70808 molecules provided CRE-recombinase by transient expression from the plasmid molecules.

In the case of the vector useful for Agrobacterium-mediated transformation, the CRE coding sequence was provided on the same vector as the selected exogenous DNA sequences (pMON73562; see FIG. 8A). In this instance, the CRE recombinase sequence was flanked by a pair of compatible lox sites which allowed for self-excision of the CRE recombinase sequence from the transferred DNA.

See Table I for various combinations of DNA molecules introduced to cells in the practice of the instant invention.

C) Preparation of Microprojectiles and Bombardment Conditions

Microprojectiles were prepared for use with the helium gun by adding 60 mg of 0.6 μm gold particles (BioRad, cat. No. 165-2262) to 1000 μl absolute ethanol and incubating, typically for at least 3 hours at room temperature followed by storage at −20° C. Twenty to thirty five μl of the sterile gold particles, or more preferably 30 to 35 μl of gold particles (30 μl contains 1.8 mg of particles), were centrifuged in a microcentrifuge for up to 1 to 5 min. The supernatant was removed and the particles carefully washed in one ml sterile water. Microprojectile particles were resuspended in about 25 μl of DNA solution containing the desired DNA molecules combined as described in Table 1. One skilled in the art would realize that the concentrations of DNA solutions may vary and that the volume of DNA used to prepare the 30 μl solution will vary with the concentration of the DNA stock and desired final concentration or desired final amount applied to recipient cells.

About 225 μl sterile water, 250 μl 2.5 M CaCl₂ and 50 μl stock spermidine (14 μl spermidine in 986 μl water; 0.1 M) were then added to the particle containing solution. The solution was then thoroughly mixed (optionally placed on ice), followed by vortexing at 4° C. for about 10 minutes and centrifugation at 500 to 700 rpm for 5 to 7 minutes.

TABLE 1
DNA molecules and concentrations used to coat particles
for bombardment transformation experiments.

| | | DNA | | | | | | |
|---|---|---|---|---|---|---|---|---|
| | | Application | | | | | | |
| Experi- | #lox | Bomb Type | | | ˜amount/ | | | ˜amount/ |
| ment | sites | or Agro | DNA | Form | treatment\hat{ } | DNA | Form | treatment\hat{ } |
| 795 | 1 | electric | lmMON55215 | linear | 100 ng | pMON55228 | plasmid | 40 ng |
| 795 | 1 | electric | pMON52215 | plasmid | 200 ng | pMON55228 | plasmid | 40 ng |
| 282 | 2 | helium | lmMON55227 | linear | 200 ng | pMON55228 | plasmid | 40 ng |
| 282 | 2 | helium | pMON55227 | plasmid | 200 ng | pMON55228 | plasmid | 40 ng |
| 2349 | 2 | electric | lmMON70811 | linear | 50 ng | MON70808 | linear | 10 ng |
| 1559 | 3 | electric | pMON70805 | plasmid | 100 ng | MON55228 | linear | 20 ng |
| 1680 | 3 | electric | pMON70805 | plasmid | 100 ng | MON55228 | linear | 20 ng |
| 2227 | 3 | electric | lmMON70805 | linear | 50 ng | MON70808 | linear | 5 ng |
| 2227 | 3 | electric | lmMON70805 | linear | 50 ng | MON70808 | linear | 10 ng |
| 2227 | 3 | electric | pMON70805 | plasmid | 50 ng | MON70808 | linear | 10 ng |
| 2252 | 3 | electric | lmMON70805 | linear | 25 ng | MON70808 | linear | 5 ng |
| 2252 | 3 | electric | lmMON70805 | linear | 25 ng | MON70808 | linear | 10 ng |
| 2453 | 3 | Agro | pMON73562 | Plasmid | NA | pMON73562 | T-DNA | NA |

The supernatant was removed and the pellet resuspended in 600 μl absolute ethanol. Following centrifugation at 500 to 700 rpm for 5 to 7 minutes, the pellet was resuspended in 36-38 μl of absolute ethanol, vortexed for approximately 20 seconds, and sonicated for 10-30 seconds. At this stage the particles were typically allowed to sit for 0-5 minutes, after which 5-10 μl of the supernatant was removed and dispensed on the surface of a flyer disk and the ethanol was allowed to dry completely. Alternatively, particles may be removed directly after resuspension and vortexing 10 to 30 seconds in about 36 μl-38 μl of ethanol, placed on the flyer disk and allowed to dry as done for the settled treatment. The bombardment chamber was then evacuated to approximately 28 in. Hg prior to bombardment. The DNA coated particles were then used for bombardment of maize cells by a helium blast of approximately 1100 psi using the DuPont Biolistics PDS1000He particle bombardment device.

Microprojectiles were prepared for use with the electric gun by suspending 10 mg of 0.6 μm gold particles (BioRad) in 50 μL buffer (150 mM NaCl, 10 mM Tris-HCl, pH 8.0). DNA was added to the suspension of gold particles as per Table 1 and gently vortexed for about five seconds.

Seventy five μL of 0.1M spermidine was added and the solution vortexed gently for about 5 seconds. Seventy five μL of a 25% solution of polyethylene glycol (3000-4000 molecular weight, American Type Culture Collection) was added and the solution was gently vortexed for five seconds. Seventy five μL of 2.5 M CaCl₂ was added and the solution vortexed for five seconds. Following the addition of CaCl₂, the solution was incubated at room temperature for 10 to 15 minutes. The suspension was subsequently centrifuged for 20 seconds at 12,000 rpm (Sorval MC-12V centrifuge) and the supernatant discarded. The gold particle/DNA pellet was washed twice with one ml 100% ethanol and resuspended to a total volume of 10 ml in 100% ethanol. The gold particle/DNA preparation was stored at −20° C. for up to eight weeks.

DNA was introduced into maize cells using the electric discharge particle acceleration gene delivery device (U.S. Pat. No. 5,015,580 incorporated herein by reference). The gold particle/DNA suspension was coated on Mylar sheets (Du Pont Mylar polyester film type SMMC2, aluminum coated on one side, over coated with PVDC co-polymer on both sides, cut to 18 mm square) by dispersion of 310 to 320 μl of the gold particle/DNA suspension on a sheet. After the gold particle suspension settled for one to three minutes, excess ethanol was removed and the sheets were air dried. Microprojectile bombardment of maize tissue was conducted as described in U.S. Pat. No. 5,015,580, incorporated herein by reference. AC voltage may be varied in the electric discharge particle delivery device. For microprojectile bombardment of H99 pre-cultured immature embryos, 30% to 40% of maximum voltage was preferably used. Following microprojectile bombardment, tissue was cultured in the dark at 27° C.

### Example 2

Transformation of H99 Immature Embryo or Callus and Selection with Paromomycin

All molecules used for transformation comprised a promoterless NPTII sequence. The NN03 target site in the recipient genome contained a 35S promoter designed such that integration into the NN03 lox site would operably link a selected DNA to the promoter sequence. The recovery of paromomycin resistant calli indicated that site directed integration occurred, operably linking the promoterless NPTII gene of the linear or circular site directed molecule with the 35S promoter of the NN03 target site. One skilled in the art would realize that a number of promoterless selection genes may be used for site-directed integration as described in the instant invention. For example, herbicide resistance genes such as glyphosate-resistant EPSPS genes or gluphosinate-resistant bar or pat genes may be employed. One of skill would also realize that while the examples presented here discuss the use of promoterless genes which are operably linked to a promoter via the targeted insertion event occurring in the host genome, target sites lacking promoters may be prepared and used for targeted insertion of a gene operably linked to a functional promoter.

Maize immature embryos (1.2-3.0 mm, 10-14 days post pollination) were isolated from greenhouse grown H99 plants comprising the NN03 target site. Preferably plants that are homozygous NN03 are backcrossed to H99 in either direction. Immature embryos were cultured on 211V medium (1× N6 basal salts, 1 mg/L 2,4-D, 1 mg/L thiamine, 0.5 mg/L nicotinic acid, 0.91 g/L L-asparagine monohydrate, 100 mg/L myo-inositol, 500 mg/L MES, 1.6 g/L MgCl2 6H2O, 100 mg/L casein hydrolysate, 0.69 g/L proline, 20 g/L sucrose; supplement with 16.9 mg/L silver nitrate; pH to 5.8 and solidify with 2 g/L Gelgro) in the dark at approximately 27° C. Immature embryos were bombarded 0-6 days after isolation. Prior to bombardment, the immature embryos were transferred to 211SV medium (medium 211 containing 12% sucrose) for 3-6 hours. Following bombardment, tissue cultures were incubated overnight and transferred to 211V medium for approximately 1 week. Following this, tissues were transferred to 211T medium (100 mg/L paromomycin) for approximately 2-3 weeks. Tissues were then transferred to 211L medium (500 mg/L paromomycin). Every 2-3 weeks, callus was subdivided into small pieces (approximately 2-4 mm in diameter) and transferred to fresh selection medium (211L; 500 mg/L paromomycin). This subculture step was repeated at 2-3 week intervals for up to about 3-15 weeks post-bombardment, typically 6 to 9 weeks, with subdivision and visual selection for healthy, growing callus.

Alternatively, immature embryos were cultured to produce embryogenic callus that was used for bombardment. Embryogenic callus was expanded and maintained by subculturing at 2-3 week intervals to fresh 211 medium. Prior to bombardment, embryogenic callus (subdivided in approximately 2-4 mm clumps) or, preferably cultured embryos, was transferred to 211 medium containing 12% sucrose (211S) for 3-6 hours. As described above for immature embryos, the bombed or Agrobacterium treated callus was transferred to media with increasing amounts of paromomycin to select for transformed tissue.

### Example 3

Transformation of Hi-II Immature Embryos or Callus

One skilled in the art would realize that site-directed integration of linear or circular molecules may be carried out in a variety of maize genotypes wherein the maize genotype used for transformation comprises at least a single target lox site. The genotype Hi-II is an example of a genotype that is useful with the current invention.

Immature embryos (1.2-3.0 mm in length) of the corn genotype Hi-II are excised from surface-sterilized, greenhouse-grown ears of Hi-II 9 to 16 days post-pollination, preferably 10-12 days post-pollination. The Hi-II genotype was developed from an A188 x B73 cross (Armstrong et al., Maize Genetics Coop Newsletter, 65:92-93, 1991, incorporated herein by reference). Approximately 30 embryos per petri dish are plated axis side down (that is, scutellar side up) on a modified N6 medium containing 1 mg/L 2,4-D, 100 mg/L casein hydrolysate, 2.9 g/L L-proline, 16.9 mg/L silver nitrate, 2 mg/L L-glycine, and 2% sucrose solidified with 2 g/L Gelgro, pH 5.8 (201V medium). An alternative modified N6 medium that may be used is 211 with appropriate supplements (e.g., hormones, sugars, vitamins, amino acids, etc.). Embryos are cultured in the dark for 2 to 6 days at 26-28° C.

Approximately 3-4 hours prior to bombardment, embryos are transferred to the above culture media with the sucrose concentration increased from 2% up to 12% (media 201SV). When embryos are transferred to the high osmoticum medium they are arranged in nickel-sized, concentric circles on the plate, starting 1 cm from the center of the dish, positioned such that they are scutellar side up and their coleorhizal end is orientated toward the center of the dish. Usually one concentric circle is formed with 25-35 embryos per plate, although it is also possible to prepare a plate with two circles of embryos.

The plates containing embryos are placed on the third shelf from the bottom, 5 cm below the stopping screen. The 1100 psi rupture discs are used for bombardment. Each plate of embryos is bombarded once with the DuPont Biolistics PDS1000He particle gun. Following bombardment, embryos are allowed to recover on high osmoticum medium (201SV, 12% sucrose) overnight (16-24 hours) and are then transferred to the appropriate selection medium

For glyphosate selection, embryos are maintained in the dark at 26° to 28° C. and typically form Type II callus during the selection process. After bombardment, embryos are allowed to incubate on media 201V for 1 to 7 days. Following this delay, the tissue is transferred to media 201V, containing 1 mM glyphosate. After approximately 2 weeks, tissues are transferred to fresh 201K selection media (supplemented with 3 mM glyphosate). After approximately 2-6 more weeks, calli are transferred to fresh 201K media. Subsequent rounds of transfers are carried out approximately every 2 weeks onto media with 3 mM glyphosate, for a total of 12-16 weeks of selection. Southern, Northern, TaqMan™, PCR, RT-PCR, or other types of molecular techniques, can then be used for analysis of transformants and of gene expression.

For paromomycin selection, embryos are maintained in the dark at 26° to 28° C. and typically form Type II callus during the selection process. After bombardment, embryos are allowed to incubate on media 211V (or 201V) for 1 to 7 days. After this delay on the initial selection plate, tissue is transferred to 211HV media with 25 mg/L paromomycin. Approximately 2 weeks later, tissue is transferred to media 211G supplemented with 50 mg/L paromomycin. After approximately another 2 weeks, the tissue is transferred to media 211T containing 100 mg/L paromomycin. After approximately 2-4 weeks, the tissue is transferred to fresh 211T media. A total of 7-15 weeks selection is typically sufficient, followed by regeneration of plants (see Example 4). Kanamycin selection may be performed in a similar manner. Following a delay period on media 211V (or 201V), tissue is transferred to media 211EE (100 mg/L kanamycin). After approximately 2 weeks, tissue is transferred to media 211F. (200 mg/L kanamycin) for a period of 2-4 weeks. Tissue is then transferred to fresh 211F for an additional 2-4 weeks. A total of 7-15 weeks selection is typically sufficient, followed by regeneration of plants (see Example 4). One of skill in the art would also recognize that media supplemented with a mix of kanamycin and paromomycin may also be used for this selection scheme. Southern, Northern, TaqMan™, PCR, RT-PCR, or other types of molecular techniques, are then be used for analysis of transformants and of gene expression.

### Example 4

Agrobacterium-Mediated Transformation

Vector pMON73562 was introduced into H99 maize comprising an NN03 target site using methods known to those of skill in the art. Agrobacterium transformed to harbor vector pMON73562 was applied to maize callus and the bacteria and maize tissue allowed to inoculate approximately 30 minutes followed by a dessicating co-culture incubation of 1-3 days (Publication No. WO0034491A2; incorporated herein by reference). These steps allow for T-DNA transfer. Following the co-culture step, the maize tissue was placed onto selection medium comprising paromomycin for selection of the plant cells comprising the promoterless NPTII inserted into the genome and operably linked to the 35S promoter of the target site. Following periodic transfers at approximately 2-3 week intervals to fresh medium supplemented with increasing amounts of paromomycin, plantlets resistant to the antibiotic were recovered.

### Example 5

Regeneration of Fertile Transgenic Plants

Fertile transgenic plants were produced from transformed H99 maize cells. Transformed callus was transferred to maturation medium 217 (N6 salts, 1 mg/L thiamine-HCl, 0.5 mg/L niacin, 3.52 mg/L benzylaminopurine, 0.91 mg/L L-asparagine monohydrate, 100 mg/L myo-inositol, 0.5 g/L MES, 1.6 g/L MgCl₂-6H₂O, 100 mg/L casein hydrolysate, 0.69 g/L L-proline, 20 g/L sucrose, 2 g/L GELGRO™, pH 5.8) for five to nine days in the dark at 26′-28° C., whereupon somatic embryos mature and shoot regeneration begins. Tissue was transferred to medium 127T (MS salts, 0.65 mg/L niacin, 0.125 mg/L pyridoxine-HCl, 0.125 mg/L thiamine-HCl, 0.125 mg/L Ca pantothenate, 150 mg/L L-asparagine, 100 mg/L myo-inositol, 10 g/L glucose, 20 g/L L-maltose, 100 mg/L paromomycin, 5.5 g PHYTAGAR™, pH 5.8) for shoot development. Tissue on medium 127T was cultured in the light at 400-600 lux at 26° C. Plantlets were transferred to soil about 3 to 6 weeks after transfer to 127T medium when the plantlets were about 3 inches tall and had roots. Plantlets were grown further in a growth chamber and fully matured in a greenhouse.

Fertile transgenic plants are also produced from transformed Hi-II maize cells. Regeneration of plants is initiated by transfer of callus from the final selection media to MS medium containing 0.04 mg/L NAA and 3 mg/L BAP (medium 105). Tissue is cultured in the dark for two weeks, followed by two weeks of culture on fresh medium 105 in low light. Tissue is subsequently transferred to MS medium with 6% sucrose without growth regulators (medium 110) and cultured in low light for approximately 2 weeks. Tissue is then subcultured to 110 medium in PHYTATRAYS™ or PLANTCONS®. Tissue in PHYTATRAYS™ or PLANTCONS® is grown under high light in a growth chamber. Regenerated plants are transferred from PHYTATRAYS™ or PLANTCONS® to soil when the plantlets are about 3 inches tall and have roots. Plantlets are grown further in a growth chamber or greenhouse. One skilled in the art would know that alternative media and selection screens may be employed to regenerate transgenic Hi-II and H99 plants.

### Example 6

Site Directed Insertion of Linear or Circular DNA Molecules into A Maize Genome

Molecular biology techniques were used to analyze the insertion events to determine if the linear or circular molecules comprising one, two or three lox sites integrated into the NN03 site in maize background H99. FIGS. 1C, 2C, 3C and 4C provide illustrations of the insertion products or predicted insertion products of a single linear DNA molecule comprising a first single lox site (MON55215), a first and second single lox site (MON55227 or MON70811) or a first, second and third single lox site (MON70805) into the NN03 lox66 target site. FIG. 8C provides an illustration of the insertion products or predicted insertion products of a single linear DNA molecule comprising a first, second and third single lox site (MON73562) delivered to the cell by Agrobacterium for targeted insertion into the NN03 lox66 target site. Southern blot technology as well as PCR methodologies were employed to determine that the linear or circular DNA molecules were integrated into a target lox site in maize genomic DNA.

One lox site: Embryos transformed by bombardment with pMON55215 or 1mMON55215 DNA molecules resulted in the production of plantlets exhibiting paromomycin resistance. Southern and PCR analysis indicated that the plants had at least one copy of the linear or circular DNA molecule comprising one lox site inserted into the target site in the genome.

Two lox sites: Embryos transformed with 1mMON70811 by bombardment resulted in the production of a plantlet exhibiting kanamycin resistance. Southern and PCR analysis indicated that the plant lacked the CRE recombinase gene and had at least one copy of the linear DNA molecule comprising 2 lox sites inserted into the target site in the genome.

Three lox sites: Embryos transformed with 1mMON70805 or pMON70805 DNA molecules resulted in the production of plantlets which exhibited paromomycin resistance. Southern and PCR analysis indicated that the plants lacked the CRE recombinase gene and had at least one copy of the linear or circular DNA molecule comprising 3 lox sites inserted into the target site in the genome.

Additionally, transformation of maize callus with pMON73562 by Agrobacterium-mediated transformation resulted in the production of paromomycin resistant plantlets, indicating that the circular molecule comprising three lox sites was targeted to the NN03 insertion site by this DNA delivery method. Southern blot analysis was used to confirm that NPT II sequences were inserted into the plant, and amplification and sequencing of the junction showed that the insertion event was targeted to the NN03 lox target site.

What is claimed is:

1. A method of integrating exogenous DNA into the genome of a eucaryotic organism comprising at least one recombination site, wherein said method comprises contacting said genome with a DNA molecule comprising said exogenous DNA and at least one site-specific recombination site which is compatible with at least one site-specific recombination site in the genome of said organism in the presence of a recombinase for integrating said exogenous DNA into the genome of said organism without removal of DNA from said organism, wherein said DNA molecule is a linear DNA molecule comprising one or more site-specific recombination sites or a circular DNA molecule comprising three or more site-specific recombination sites.

2. A method according to claim 1 further comprising identifying a transgenic recipient cell of said organism having said exogenous DNA integrated into its genome.

3. A method according to claim 2 further comprising regenerating a fertile transgenic organism from said identified transgenic recipient cell.

4. The method of claim 1, wherein said DNA molecule is a linear DNA molecule comprising one site-specific recombination site.

5. The method of claim 1, wherein said DNA molecule is a linear DNA molecule comprising two site-specific recombination sites.

6. The method of claim 1, wherein said DNA molecule is a linear DNA molecule comprising three site-specific recombination sites.

7. The method of claim 1, wherein said DNA molecule is a circular DNA molecule comprising three site-specific recombination sites.

8. The method of claim 1, wherein at least one said site-specific recombination site is selected from the group consisting of a lox site, a gix site, an RS site and a frt site.

9. The method of claim 8, wherein at least one said site-specific recombination site is a lox site.

10. The method of claim 1, wherein said DNA molecule is a single-stranded molecule.

11. The method of claim 1, wherein said DNA molecule is a double-stranded molecule.

12. The method of claim 1 wherein said recombinase is provided to said recipient cell as a DNA molecule, RNA molecule or protein molecule.

13. The method of claim 1 wherein said recombinase is provided by crossing said transgenic organism comprising said exogenous DNA with a second transgenic organism comprising a recombinase.

14. The method of claim 3 wherein said transgenic organism is a plant.

15. The method of claim 1, wherein the contacting comprises a transformation method selected from the group consisting of microparticle bombardment, PEG-mediated transfer, electroporation and Agrobacterium-mediated transformation.

16. An isolated linear DNA molecule comprising one, two or three or more site specific recombination sites suitable for use with a recombination method of claim 15.

17. An isolated linear DNA molecule of claim 16, wherein at least one of the site-specific recombination sites is a lox site.

18. A circular DNA comprising three or more site-specific recombination sites suitable for use with a recombination method of claim 15.

19. A circular DNA molecule of claim 18, wherein at least one of the site-specific recombination sites is a lox site.

20. A circular DNA molecule of claim 19 wherein said molecule comprises a CRE recombinase coding sequence.

21. A circular DNA molecule of claim 18 wherein said molecule is isolated from a bacterial host or generated by in vitro amplification methods.
